(19) United States
(12) Patent Application Publication
(10) Pub. No.: US 2008/0038355 A1
(43) Pub. Date: Feb. 14, 2008

(54) BIOADHESIVE DOSAGE FORM OF STEROIDS

(75) Inventors: Chih-Chiang Yang, Taipei City (TW); Yuan-Chih Le, Taipei Hsien (TW); Chao-Cheng Liu, San Chung City (TW)

Correspondence Address: OCCHIUTI ROHLICEK & TSAO, LLP, 10 FAWCETT STREET, CAMBRIDGE, MA, 02138, US

(73) Assignee: Pharmaceutical Industry Technology and Development Center, Taipei Hsien (TW)

(21) Appl. No.: 11/876,347

(22) Filed: Oct. 22, 2007

Related U.S. Application Data
(62) Division of application No. 10/892,918, filed on Jul. 16, 2004.

(51) Int. Cl. A61K 9/00; A61K 31/56; A61P 43/00

(52) U.S. Cl. 424/488; 514/178

(57) ABSTRACT

Bioadhesive steroid nanoparticles are used to prepare a rapidly disintegrating solid oral dosage form of steroid. The bioadhesive steroid nanoparticles have a bioadhesive polymer surrounding the steroid nanoparticles and with an average diameter of less than 1000 nm. Hence, the solid oral dosage form, containing the bioadhesive steroid nanoparticles, a disintegrant and an effervescent agent, instantly disintegrates in the oral cavity and adheres on the periodontal pocket and oral mucosa to release steroid rapidly.

### BACKGROUND OF THE INVENTION

1. Field of Invention

The present invention relates to a dosage form to remedy oral disease. More particularly, the present invention relates to a rapidly disintegrating solid oral dosage form comprising a bioadhesive polymer to deliver steroids to the periodontal pocket and oral mucosa.

2. Description of Related Art

There is an increasing interest in developing improved drug delivery devices for the periodontal pocket and oral mucosa in treatment of oral diseases such as oral cancer, periodontal disease, and oral herpes. It is well known that many steroids, such as triamcinolone acetonide, hydrocortisone, betamethasone, dexamethasone, flumethasone, prednisolone, isoflupredone, methylprednisolone, prednisolone, and predisone, are anti-inflammatory drugs and have proved particularly useful in the treatment of dermatological conditions. For example, triamcinolone acetonide has been proved to have marked efficacy in the treatment of dermatosis, eczema, neurodermitis, impetigo, psoriasis, pruritis and other related diseases; a therapy method for oral herpes simplex is described in U.S. Pat. No. 4,466,956.

These anti-inflammatory steroids are often used in nasal sprays and topical creams. Treating intraoral disease with sprays or creams is not very convenient or sanitary, since a spray nozzle or a finger has to be inserted into the oral cavity. Moreover, the amount of the spray or cream used is difficult to control, and the drug dosage applied is consequently uncertain. Other problems include difficulty in carrying and storing sprays and creams.

### SUMMARY OF THE INVENTION

In one aspect, the present invention provides a rapidly disintegrating solid oral dosage form of steroids and a method of forming the same, so that the steroids can rapidly disintegrate and be dissolved in the oral cavity.

In accordance with the foregoing and other aspects of the present invention, a rapidly disintegrating solid oral dosage form of steroids comprising steroid granules distributed in a solid matrix is provided. The steroid granules comprise steroid nanoparticles dispersed in a bioadhesive polymer matrix and a granulating agent coating on the bioadhesive polymer matrix. The solid matrix comprises a disintegrant, an effervescent agent and an excipient.

A method of forming a rapidly disintegrating solid oral-dosage form of a steroid comprises the following steps. First, a steroid, a surfactant and water are mixed to form a micelle solution containing steroid micelles. The micelle solution is then added in drops to a bioadhesive polymer aqueous solution to form a suspension solution, so that the bioadhesive polymer surrounds the steroid to form bioadhesive steroid nanoparticles having a diameter less than 1000 nm. The suspension solution is then concentrated and combined with a granulating agent to form granules. Finally, the granules are combined with a lubricant, an excipient, a disintegrant, and an effervescent agent to form tablets.

In the forgoing, the bioadhesive polymer comprises chitosan, alginate, cellulose or gelatin. The surfactant comprises polyoxyethylene alkyl ethers, soya lecithin, polyoxyethylene sorbitan monooleate, or a sorbitan fatty acid ester. The granulating agent comprises lecithin and starch.

Once the steroid tablet is put into the oral cavity, the tablet rapidly disintegrates in a very short time to release the steroid nanoparticles, which adhere to the periodontal pocket and oral mucosa.

It is to be understood that both the foregoing general description and the following detailed description are made by examples and are intended to provide further explanation of the invention as claimed.

### BRIEF DESCRIPTION OF THE DRAWINGS

The invention can be more fully understood by reading the following detailed description of the preferred embodiment, with reference made to the accompanying drawings as follows:

FIG. 1 shows the particle size distribution of bioadhesive triamcinolone acetonide nanoparticles in a suspension solution; and

FIG. 2 shows the dissolution rate over time for a rapidly disintegrating tablet containing bioadhesive triamcinolone acetonide nanoparticles according to the present invention.

### DESCRIPTION OF THE PREFERRED EMBODIMENTS

A tablet dosage form of steroids would resolve the problem described above; however, no tablet dosage form of steroids has been developed, since the slight water solubility of steroids is a problem in the use thereof. Therefore, there is a need to develop a dosage form of steroids to deliver the drug to the intraoral target tissue and let the drug become rapidly available for the oral cavity. The present invention provides a rapidly disintegrating solid oral form of steroids comprising a bioadhesive to deliver the steroids to the periodontal pocket and oral mucosa.

Preparation of Granules Containing a Steroid

A rapidly disintegrating tablet containing a steroid is prepared by the following method. First, a steroid and a surfactant is added into a tri-pyrophosphate aqueous solution and then is stirred to prepare Solution I. In solution I, the steroid is surrounded by the surfactant to form micelles and is dispersed in solution I, and the tri-pyrophosphate remains in the water phase. The suitable surfactant is, for example, polyoxyethylene alkyl ethers, soya lecithin, polyoxyethylene sorbitan monooleate, or a sorbitan fatty acid ester.

Next, the Solution I is added in drops into Solution II, which is an aqueous acid solution of chitosan, and then is stirred to prepare a suspension solution. Chitosan is known as a bioadhesive (U.S. Pat. No. 5,993,846). In the suspension solution, chitosan surrounds the steroid micelles, and the tri-pyrophosphate, acting as a cross-linking reagent, induces the polymerization of the chitosan to form steroid nanoparticles in the solution. Chitosan acid solution can also be replaced by other hydrogel solution, such as solutions of alginate, cellulose or gelatin. If the alginate solution is used as Solution II, the tri-pyrophosphate aqueous solution, in preparing Solution I, is replaced with CaCl₂ aqueous solution.

The nanoparticles in the suspension solution are then granulated in a standard wet granulation process. First, the suspension solution is concentrated, for example, under a reduced pressure to about ¼-⅛ of the original volume. Lecithin powder is subsequently added into the concentrated suspension solution and continually mixed to form a homogeneous mixture. Next, starch is added into the homogeneous mixture to form granules. The granules containing steroids are dried in an oven and then milled. In the steroid granules, the steroid nanoparticles are dispersed in the matrix of the bioadhesive polymer.

For example, granules containing triamcinolone acetonide were prepared by following the method described above, and the amounts used for each component are listed in the Table 1. If the desired particle size of triamcinolone acetonide is smaller, the added amount of the Cemophor RH40 has to be large enough to disperse triamcinolone acetonide nanoparticles.

TABLE 1

| Solution I | | |
|---|---|---|
| Triamcinolone acetonide | 20 | mg |
| Cemophor RH40 | 2-20 | mg |
| Tri-pyrophosphate | 0.35 | mg |
| H₂O | 199.65 | mg |
| Solution II | | |
| Chitosan | 1.45 | mg |
| Acetic acid or lactic acid | 0.35 | mg |
| H₂O | 498.2 | mg |
| Granulating agent | | |
| Lecithin | 2 | g |
| Starch | 80 | g |

Preparation of Tablets Containing Steroid Granules

After milling, the granules are lubricated with a lubricant and mixed with an excipient, an effervescent agent and a disintegrant to be made into tablets by a tablet machine. Any pharmaceutically acceptable lubricant, excipient, effervescent agent and disintegrant can be used here. The disintegrant comprises crospovidone, croscermellose sodium, microcrystalline cellulose, Methylcellulose, magnesium aluminum silicate, calcium carboxymethyl cellulose, or sodium carboxymethylcellulose. The Effervescent agent is, for example, citric acid/Na₂CO₃, tartaric acid/Na₂CO₃, or tartaric acid/sodium citrate. For example, tablets containing triamcinolone acetonide granules were prepared by the method described above, and amounts used for each component are listed in Table 2.

TABLE 2

| | | |
|---|---|---|
| Granules containing triamcinolone acetonide | 15 | g |
| Excipient | | |
| Mannitol | 3 | g |
| Disintegrant | | |
| Crospovidone | 2 | g |
| Lubricant | | |
| Mg stearate | 0.1 | g |
| Aerosil | 0.1 | g |
| Effervescent agent | | |
| Citric acid + Na₂CO₃ | 1 | g |

Analysis

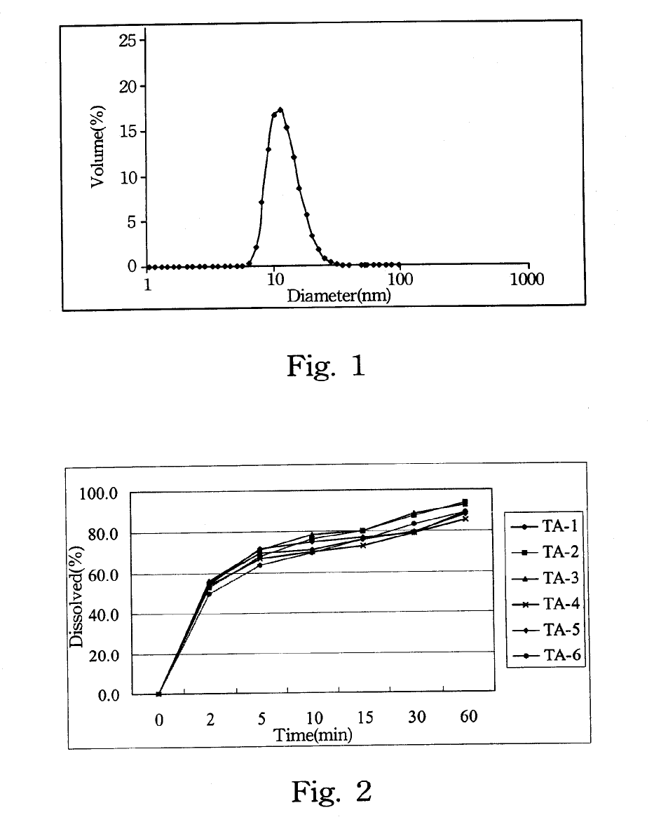

The particle size of the chitosan surrounding the triamcinolone acetonide nanoparticles, in the suspension solution described above, were analyzed by a nanoparticle size analyzer (Nanoseries Zetasizer, Malvern, U.K.), and the results of analysis are listed in FIG. 1. As shown in FIG. 1, the particle size is quite centralized around 10-50 nm and most of the nanoparticles are smaller than 100 nm.

The disintegration times of the tablet containing the triamcinolone acetonide granules in water were tested and are listed in Table 3. In Table 3, the average disintegration time is only about 18.7 sec.

TABLE 3

No.

| | TA-1 | TA-2 | TA-3 | TA-4 | TA-5 | TA-6 |
|---|---|---|---|---|---|---|
| Time (sec) | 14 | 16 | 16 | 20 | 22 | 24 |

| | |
|---|---|
| Avg (sec) | 18.7 |
| STD (sec) | 3.9 |

The dissolution times of the tablet containing the triamcinolone acetonide granules in water were analyzed, and the results are shown in FIG. 2. In FIG. 2, all tablets were at least 50% dissolved at 2 minutes and almost completely dissolved at 60 minutes.

In light of the forgoing, the preferred embodiment of the invention provides a rapidly disintegrating tablet dosage form of steroids to rapidly deliver the drug to the periodontal pocket and oral mucosa. In this tablet dosage form, the steroid nanoparticles are dispersed in a matrix of a bioadhesive polymer and then granulated. The steroid granules are subsequently mixed with some lubricant, disintegrant, effervescent agent and excipient to form a tablet. Hence, when the steroid tablet is administered in the oral cavity, the tablet rapidly disintegrates in a very short time to release the steroid nanoparticles, which adhere to the periodontal pocket and oral mucosa.

For example, the triamcinolone acetonide tablet disintegrates in a period of less than 25 seconds. Due to the small size, about 10-50 nm, of the bioadhesive triamcinolone acetonide nanoparticles, about 50% of the triamcinolone acetonide is dissolved within 2 minutes. This means that about 50% of the triamcinolone acetonide can be released within 2.5 minutes after the tablet administered in the oral cavity to treat an oral disease.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A rapidly disintegrating solid oral dosage form of steroid, the dosage form comprising:
a solid matrix comprising a disintegrant, an effervescent agent and an excipient; and
steroid granules distributed in the solid matrix, comprising steroid nanoparticles dispersed in a matrix of an bioadhesive polymer.

2. The dosage form of claim 1, where in the steroid is selected from the group consisting of triamcinolone acetonide, hydrocortisone, betamethasone, dexamethasone, flumethasone, prednisolone, isoflupredone, methylprednisolone, prednisolone, and predisone.

3. The dosage form of claim 1, wherein the bioadhesive polymer is selected from a group consisting of chitosan, alginate, cellulose and gelatin.

4. The dosage form of claim 1, wherein the surfactant is selected from the group consisting of polyoxyethylene alkyl ethers, soya lecithin, Polyoxyethylene sorbitan monooleate, and sorbitan fatty acid esters.

5. The dosage form of claim 1, wherein the granulating agent comprises lecithin and starch.

6-14. (canceled)
